(19) United States
(12) Patent Application Publication
(10) Pub. No.: US 2012/0179085 A1
(43) Pub. Date: Jul. 12, 2012

(54) Device and Process for Creation of Active Antimicrobial Iodine from Inactive Iodine

(76) Inventor: James C. Richards, Sudbury, MA (US)

(21) Appl. No.: 13/347,050

(22) Filed: Jan. 10, 2012

Related U.S. Application Data
(60) Provisional application No. 61/431,123, filed on Jan. 10, 2011.

(51) Int. Cl. A61M 35/00; B05D 5/00

(52) U.S. Cl. 604/3; 427/2.1

(57) ABSTRACT

A device and process that creates active molecular iodine on a swab. The device has two chambers sealed from one-another. One of the chambers contains a swab. Each chamber contains a different reagent such that when the device is broken or crushed to activate it, the contents of both chambers mix on the swab. The reagents are such that, when mixed, they produce molecular iodine on the swab which acts as a topical antiseptic. The reagents can be an iodide in one chamber and an iodate in the second chamber with a buffer. Alternatively one of the chambers can contain dry molecular iodine in contact with the swab. Finally, one of the chambers can contain molecular iodine in a solution of ethanol, while the other can contain a aqueous solution of a buffer to maintain a proper pH.

This application is related to and claims priority from U.S. Provisional Patent application No. 61/431,123 filed Jan. 10, 2011. Application 61/431,123 is hereby incorporated by reference.

### BACKGROUND

1. Field of the Invention

The present invention relates generally to antimicrobial solutions and more particularly to a device and process for creation of active antimicrobial iodine from inactive iodine forms.

2. Description of the Prior Art

Molecular iodine or I₂ is the dominant antimicrobial form of iodine in aqueous solution. Unfortunately, I₂ undergoes hydrolysis in aqueous solution according equations (1) and (2) known in the art:

I₂+H₂O<->HOI+H⁺−I hydrolysis of molecular iodine (1)

3I₂+3H₂O<->IO₃⁻+5I⁻+6H⁺ iodate formation (2)

In any iodine-containing aqueous solution, I₂ is the dominant antimicrobial iodine compound. Therefore, the most potent and least potentially toxic iodine formulation contains predominately i.e., >95% I₂. An aqueous solution containing predominately I₂ would be a potent antibacterial, antiviral and antifungal solution that would kill pathogens on contact, possess little or no cytotoxicity, and have minimal color and odor (these properties are present in concentrations above 50 μg/mL I₂). An aqueous solution of I₂ is an ideal commercial product for many antibacterial applications including disinfection of skin epidermis including general epidermis, inside the nasal passages, groin, perineum, axilla, between fingers and toes, inside outer ear and mucous membranes including oral cavity, vagina, anus and inner nasal passages.

Others have tried and failed to produce a stable solution of aqueous I₂ at a concentration of I₂ >95%. Commercial prior art products containing I₂ also contain very large amounts of other iodine compounds that can produce tissue toxicity, have pronounced odor, color and staining properties. The ratio of I₂ to other iodine compounds is greatest in Lugol's solution known in the art which contains greater than equal molar amounts of other non-active iodine compounds. Commercial preparations containing iodine compounds in addition to I₂ include:

1. Iodine Topical Solution, an aqueous solution containing 2% iodine and 2.4% sodium iodide;

2. Strong Iodine Solution (Lugol's Solution), an aqueous solution containing 5% iodine and 10% potassium iodide;

3. Iodine Tincture, containing 2% iodine and 2.4% sodium iodide aqueous ethanol (1:1);

4. Strong Iodine Tincture containing 7% iodine and 5% sodium iodide in 95% ethanol.

Other commercial iodine-containing preparations predominately include iodine compounds in combination with complexing agents including iodophores, which represent the combination of iodine with a carrier of high molecular weight. Some of these include polyvinyl pyrrolidone, polyether glycols, polyvinyl alcohols, polyacrylic acid, polyamides, polyoxyalkylenes and polysaccharides. Iodine binds tightly to these carriers making very little molecular I₂ is available i.e., 3-5 μg/mL I₂. For example, 10% povidone-iodine (Betadine®) contains 3-5 μg/mL I₂ and 10,000 μg/mL of other iodine compounds. It would be advantageous to have an antimicrobial iodine solution that contains only I₂, and therefore has the lowest possible amount of iodine that might cause cytotoxicity but the highest concentration of active, antimicrobial iodine. A solution containing >95% I₂ would have the highest possible therapeutic index of any antimicrobial iodine composition.

### SUMMARY OF THE INVENTION

The present invention relates to a device and process that creates active molecular iodine on a swab. The device has two chambers sealed from one-another. One of the chambers contains a swab. Each chamber contains a different reagent such that when the device is broken or crushed to activate it, the contents of both chambers mix on the swab. The reagents are such that, when mixed, they produce molecular iodine on the swab which acts as a topical antiseptic. The reagents can be an iodide in one chamber and an iodate in the second chamber with a buffer. Alternatively one of the chambers can contain dry molecular iodine in contact with the swab. Finally, one of the chambers can contain molecular iodine in a solution of ethanol, while the other can contain a aqueous solution of a buffer to maintain the pH between approximately 4-6.

### DESCRIPTION OF THE FIGURE

Attention is directed to at least one figure that illustrates features important to the present invention:

FIG. 1 shows the two-chamber device of the present invention.

### DESCRIPTION OF THE INVENTION

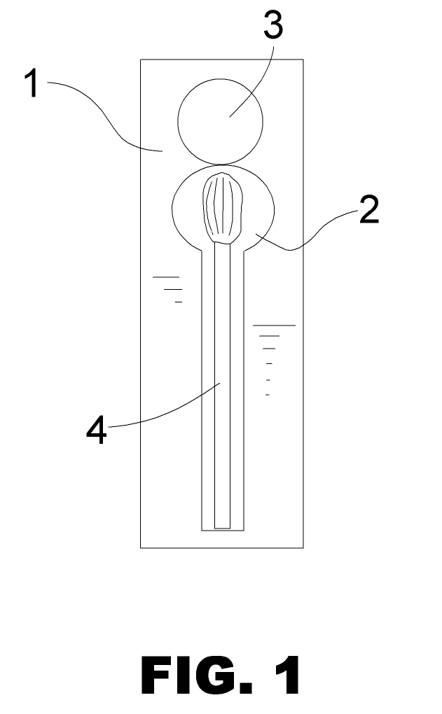

The present invention relates to a device and process that both facilitates creation of I₂ immediately before use by creating active antimicrobial I₂ from inactive iodine compounds and also provides an applicator or delivery device for applying the newly created active I₂ to solid surfaces such as skin and mucous membranes. A preferred delivery device is a swab with an absorbent head attached to the end of a rigid stick. The most general form of the device is a package 1 shown in FIG. 1 with one side composed of a clear polymer and the reverse side composed of a friable material such as metal foil capable of being easily opened to remove the swab. The device can have two chambers 2, 3. Chamber 1 2 can contain the delivery device composed of an absorbent swab 4 and stick. Chamber 2 3 can contain a fluid of sufficient volume to saturate the absorbent swab. The clear polymer side and the reverse side composed of friable material are bound together by for example a heat seal. Other methods known in the art such as adhesives may also be used. The space between chamber 1 and chamber 2 is sealed in a manner that permits the seal to be broken and permits the contents of chamber 2 to enter chamber 1 and be absorbed into the swab head.

At least two processes can be used to create active I₂ on a swab. Also, numerous devices can be used to mix or create a wet swab. A prior art package is shown in FIG. 1 that contains two chambers that allow mixing of chemicals onto the swab. Any device with a first and second chamber that can be broken to mix the contents onto a swab is within the scope of the present invention.

A first process or embodiment for creating active I₂ is to impregnate the absorbent swab in chamber 1 with potassium iodide at ˜pH 8-9, unbuffered. The impregnated swab is dried by for example by lyophilization. The solution in chamber 2 can contain potassium iodate in a strong acetate buffer ˜0.5 M at pH 3 as well as 0.001 N HCl. The seal between swab and fluid is broken for use, and the swab head absorbs most of the iodate solution. Molecular iodine is formed immediately on the swab head which turns light yellow as an internal positive control indicating the presence of I₂ or free iodine in aqueous solution. The process thus creates active antimicrobial I₂ for use on the swab. The user opens the package by pushing the swab on stick delivery device through the friable back or other method of removing the swab saturated with newly created I₂. The user would then swab skin or mucous membranes to kill bacteria, viruses and fungi.

A second process or embodiment for creating active I₂ is to impregnate the absorbent swab in chamber 1 with I₂ in 100% ethanol or other 100% alcohol. The swab is then dried to eliminate the alcohol and produce a dry swab containing crystals of inactive, solid molecular iodine. A solution in chamber 2 contains an acetate buffer ˜0.5 M at pH 3 as well as 0.001 N HCl. The seal Separating chamber 1 and chamber 2 is broken, and the swab head absorbs most of the acetate buffer solution. Molecular iodine is formed immediately by dissolution of the iodine crystals in the acidic solvent, and the swab head turns light yellow as an internal positive control indicating the presence of molecular iodine or free iodine in aqueous solution. The user opens the package by pushing the swab on stick delivery device through the friable back or other method of removing the swab saturated with newly created I₂. The user would then swab skin or mucous membranes to kill bacteria, viruses and fungi.

In both the first and second process, the exact amount of I₂ created from inactive iodine compounds can be predicted from the concentration of dried, inactive iodine compounds adsorbed into the swab. In both processes >95% of inactive iodine compounds are converted into active aqueous I₂.

Another way to create aqueous active molecular iodine prior to use to kill microorganisms is to use a two part configuration as previously described, but in chamber 1 have solid iodine dissolved in 100% ethanol (very small volume). In this application there is a solution of molecular iodine in 100% ethanol e.g., 1.0%. The swab is saturated with water and pH buffer e.g., pH 4-6. When the seal is broken, molecular iodine in water is created that is antimicrobial and achieves the same effect as the previously described embodiments. Typically, molecular iodine is not stable in water. In this embodiment, the device has two chambers. Chamber 1 has an aqueous solution of water and pH buffer and salt, and the saturated swab has a volume e.g., 1.0 mL. Chamber 2 has a solution of molecular iodine in 100% ethanol e.g., 1% w/v e.g., 0.01 mL, and when they are mixed by breaking a seal between the two chambers, an antimicrobial solution of molecular iodine is created at a concentration of between 10-100 ug/mL. The 100% ethanol is then effectively diluted to about 1%, and this is not irritating to mucous membranes i.e., nasal passages, throat, etc. Molecular iodine at 10-100 ug/mL is extremely effective at killing pathogens. As in other embodiment, the swab will turn yellow/orange after the seal is broken. Molecular iodine in 100% ethanol is bright red.

A 0.01 mL solution of molecular iodine in 100% ethanol is non-polar. The volume is about one drop. Consider a sphere (hydrophilic wall) containing the 0.01 mL ethanol inside the chamber with the swab. Compression of the swab/sphere combination results in rupture of the sphere and release of the molecular iodine in 100% ethanol, and results in an active solution of molecular iodine in aqueous solution in the range of 10-100 ug/mL. Again, there are two chambers; however, in this case the micro sphere is adjacent to the swab. The user breaks the sphere seal and releases solution 1 that then combines with solution 2.

A great advantage of the present invention is that molecular iodine is the dominant antimicrobial iodine composition, and molecular iodine is not stable in water/aqueous solution. Creation of aqueous molecular iodine immediately before use creates an effective antimicrobial solution and provides an effective antimicrobial solution for killing microorganisms on skin, inside nose, in the throat or any topical application.

In summary, in the preferred embodiment, a large-headed rayon swab sits inside a clear polymer package. Located near the rayon head is a second pouch containing a fluid. The device works by breaking the seal between the rayon head and the liquid. The amount of liquid is designed to be completely absorbed into the swab head which becomes saturated. The user grasps the swab stick at the bottom by breaking the foil seal on the underside and pulls the wet swab to a position half way down the device and then uses the stick to push the wet swab out through the foil. The wet swab can then be used to apply the fluid to a site. Typically, the rayon swab is impregnated with potassium iodide at ˜pH 8-9,unbuffered. The impregnated swab can be dried by for example by lyophilization. The solution phase can contain potassium iodate in strong acetate buffer ˜0.5 M at pH 3.5->4 as well as 0.001 N HCl. When the seal between swab and fluid is broken, the swab head absorbs most all of the iodate solution. Molecular iodine is formed immediately, and the swab head turns light yellow as an internal positive control indicating the presence of molecular iodine or free iodine in aqueous solution. The user can then swab the inside of each nostril, or elsewhere, and discard the used swab.

Several descriptions and illustrations have been provided to aid in understanding the present invention. One with skill in the art will realize that numerous changes and variations can be made without departing from the spirit of the invention. Each of these changes and variations is within the scope of the present invention.

I claim:

1. A method for creating active I₂ on a swab comprising:
providing an activatable sealed device having both a first and a second separated chamber, said first chamber containing a swab;
placing a first reagent in the first chamber with said swab;
placing a second reagent in the second chamber, wherein when said device is activated, the reagent from the first chamber mixes on said swab with the reagent from the second chamber producing a solution on said swab containing molecular iodine;
whereby, said molecular iodine on said swab acts as a topical antiseptic.

2. The method of claim 1 wherein said first reagent contains potassium iodide.

3. The method of claim 1 wherein said second reagent contains potassium iodate.

4. The method of claim 1 wherein said first reagent is dried molecular iodine.

5. The method of claim 1 wherein the second reagent contains a buffer.

6. The method of claim 5 wherein said second reagent also contains 0.001 N HCl.

7. The method of claim 4 further comprising: impregnating said swab with molecular iodine and ethanol; drying said swab; placing said swab in said first chamber.

8. A method for creating active molecular iodine on a swab comprising:
providing a breakable sealed device having both a first and a second separated chamber that when broken combines contents from the first and second chambers, said first chamber containing a swab;
placing an aqueous solution of a pH buffer in said first chamber with the swab;
placing a solution of molecular iodine in ethanol in said second chamber;
whereby, when said device is broken, molecular iodine appears on said swab acting as a topical antiseptic.

9. The method of claim 8 wherein said solution of molecular iodine in ethanol is 1%.

10. The method of claim 8 wherein said aqueous solution of a pH buffer has a pH of between approximately 4-6.

11. The method of claim 8 wherein when said device is broken, concentration of molecular iodine on said swab is between approximately 10-100 ug/mL.

12. An apparatus that creates active I₂ on a swab comprising:
an activatable sealed device having both a first and a second separated chamber, said first chamber containing a swab;
a first reagent in the first chamber with said swab;
second reagent in the second chamber, wherein when said device is activated, the reagent from the first chamber mixes on said swab with the reagent from the second chamber producing a solution on said swab containing molecular iodine;
whereby, said molecular iodine on said swab acts as a topical antiseptic.

13. The apparatus of claim 12 wherein said first reagent contains potassium iodide.

14. The apparatus of claim 12 wherein said second reagent contains potassium iodate.

15. The apparatus of claim 12 wherein said first reagent is dried molecular iodine.

16. The apparatus of claim 12 wherein the second reagent contains a buffer.

17. The apparatus of claim 16 wherein said second reagent also contains 0.001 N HCl.
